(19) United States
(12) Patent Application Publication
(10) Pub. No.: US 2020/0385296 A1
(43) Pub. Date: Dec. 10, 2020

(54) METHOD FOR CHARACTERIZING THE PREVALENCE OF A BIOCHEMICAL CONDITION WITHIN A POPULATION

(71) Applicant: Biobot Analytics, Inc., Cambridge, MA (US)

(72) Inventors: Mariana G. Matus Garcia, Cambridge, MA (US); Newsha Ghaeli, Cambridge, MA (US)

(21) Appl. No.: 16/893,308

(22) Filed: Jun. 4, 2020

Related U.S. Application Data
(60) Provisional application No. 62/857,216, filed on Jun. 4, 2019.

(51) Int. Cl. C02F 3/00; G01N 33/18; G05D 21/02

(52) U.S. Cl. CPC C02F 3/006; G01N 33/1866; C02F 2209/44; C02F 2209/22; C02F 2209/36; G05D 21/02

(57) ABSTRACT

A method for estimating the prevalence of a biochemical condition in a population includes accessing, based on a wastewater sample collected by a wastewater sampling system that samples from a wastewater flow serving the population: a biochemical indicator concentration of a biochemical indicator characterized by a correlation of the biochemical indicator with the biochemical condition; and a biomarker concentration of a biomarker correlated with presence of human excreta. The method also includes: normalizing the biochemical indicator concentration based on the biomarker concentration; adjusting the biochemical indicator concentration based on decay characteristics of the biochemical indicator in wastewater; calculating a biochemical indicator mass estimate for the population based on the biochemical indicator concentration and an effective flow volume of the wastewater flow; and estimating a prevalence of the biochemical condition in the population based on the correlation of the biochemical indicator to the biochemical condition and the biochemical indicator mass estimate.

### CROSS-REFERENCE TO RELATED APPLICATIONS

This application claims the benefit of U.S. Provisional Application No. 62/857,216, filed on 4 Jun. 2019, which is incorporated in its entirety by this reference.

This application is related to U.S. patent application Ser. No. 14/528,465, filed on 31 Jul. 2019, which is incorporated in its entirety by this reference.

### TECHNICAL FIELD

This invention relates generally to the field of wastewater sampling and more specifically to a new and useful method for characterizing the prevalence of a biochemical condition within a population in the field of wastewater sampling.

### BRIEF DESCRIPTION OF THE FIGURES

FIG. 1 is a flowchart representation of a method;

FIG. 2 is a schematic representation of a wastewater sampling system; and

FIG. 3 is a flowchart representation of one variation of the method.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. Method

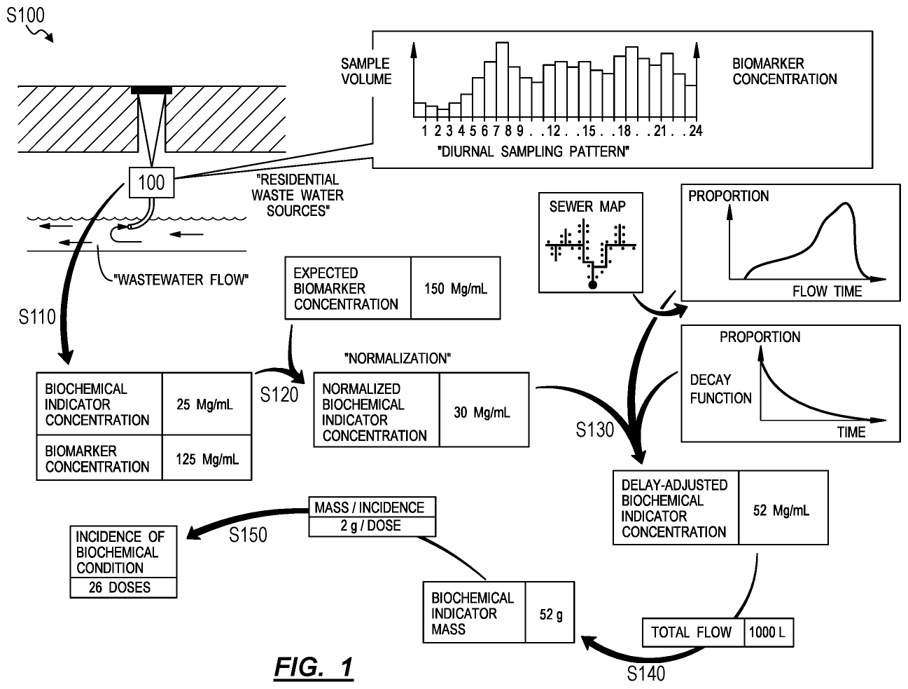

As shown in FIG. 1, a method S100 for estimating the prevalence of a biochemical condition in a population includes: accessing a biochemical indicator concentration of a biochemical indicator correlated with the biochemical condition, the biochemical indicator concentration derived from a wastewater sample collected from a wastewater flow serving the population by a wastewater sampling system; and accessing a biomarker concentration of a biomarker correlated with presence of human excreta, the biomarker concentration derived from the wastewater sample in Block S110. The method S100 also includes: normalizing the biochemical indicator concentration based on the biomarker concentration to calculate a normalized biochemical indicator concentration in Block S120; adjusting the normalized biochemical indicator concentration based on decay characteristics of the biochemical indicator in wastewater to generate a decay-adjusted biochemical indicator concentration in Block S130; calculating a biochemical indicator mass estimate for the population based on the decay-adjusted biochemical indicator concentration and an effective flow volume of the wastewater flow in Block S140; and estimating the prevalence of the biochemical condition in the population based on the correlation of the biochemical indicator to the biochemical condition and the biochemical indicator mass estimate in Blocks S150.

### 2. Applications

Generally, the method S100 is executed by a computer system cooperating with a wastewater sampling system deployed in an active sewer network upstream from a wastewater treatment facility to characterize a prevalence of a biochemical condition (e.g., drug usage, viral disease, bacterial disease) within a population served by the active sewer network. More specifically, the computer system can: access a concentration of a biochemical indicator (e.g., a drug metabolite, viral RNA) in a sample collected by the wastewater sampling system, where the biochemical indicator is correlated with the prevalence of the biochemical condition; accessing a biomarker concentration correlated with presence of human excreta (e.g., a urinary biomarkers such as creatinine, or fecal biomarkers such as gut RNA); normalizing this biochemical indicator concentration based on the biomarker concentration; adjusting the biochemical indicator concentration based on decay effected while in the active sewer system (e.g., due to bacteria consumption and/or the chemical reactivity of the biochemical indicator); estimating a mass of the biochemical indicator input to the sewer system; and calculating a prevalence of the biochemical condition (e.g., a number of drug doses ingested, a number of viral infections).

Thus, by normalizing the concentration of the biochemical indicator relative to the concentration of a biomarker and adjusting the concentration of the biochemical indicator for the time the biochemical indicator is present within the active sewer system, the computer system can generate accurate and actionable statistics on the prevalence of a biochemical condition in a population that are immune from the effects of reporting bias, thereby enabling governments and other entities to efficiently direct law enforcement and/or public health resources to address drug addiction and/or viral epidemics in a population. For example, the computer system can transform a concentration of a drug metabolite derived from a single sample of wastewater collected over a 24-hour sampling period and calculate a number of drug dosages occurring within a population with a plus-or-minus 20% accuracy in a population of 4,000 people. Additionally, by repeatedly sampling wastewater from the active sewer system and recalculating the prevalence of the biochemical condition, the computer system can generate a high-resolution time series indicating changes in the prevalence of the biochemical condition over time, further guiding the responses of law enforcement and/or public health entities.

The computer system accesses data derived from a wastewater sample collected by a wastewater sampling system, which intermittently (e.g., regularly, periodically) samples a wastewater flow at a sewage access point in a sewage network. Thus, the wastewater sampling system collects wastewater samples that contain biochemical indicators (e.g., drug metabolites or viral RNA) in human waste from any upstream source. The computer system can then associate concentrations of the biochemical indicator in wastewater samples within a geographical region corresponding to the wastewater flow at the particular sewage access point (i.e., the sampling location) based on a map of the sewer system but without direct or precise measurement of the flow rate of the wastewater flow. Furthermore, by sampling the wastewater flow upstream from a water treatment facility, the wastewater sampling system is able to collect wastewater samples before degradation of these biochemical indicators occurs due to consumption by bacteria or degradation via reactions with other chemicals in the wastewater.

Generally, an active sewer system may function as a “combined sewer system” that collects wastewater from residential, commercial, and/or natural sources (e.g., from runoff or groundwater infiltration). In a combined sewer system, the proportion of wastewater from residential sources may vary widely. The computer system can therefore execute Blocks of the method S100 to analyze wastewater samples—collected by the wastewater sampling system—proportional to a biomarker pattern (e.g., a diurnal biomarker pattern) over a 24-hour period, thereby compensating for variance in the proportion of wastewater from residential sources and eliminating reliance on direct wastewater flow measurements or complex water flow estimates for residential sources to derive accurate insights from the wastewater sample.

For example, the wastewater sampling system can collect a set of samples—such as separated by a fixed time interval—of equal volume over a 24-hour period. The computer system can then analyze these samples to calculate the concentration of a biomarker in each sample. The wastewater sampling system and the computer system can cooperate to repeat this process over multiple days to establish a diurnal biomarker pattern. Once the computer system calculates a diurnal biomarker pattern, the wastewater sampling system can collect samples of wastewater such that the volume of each subsample comprising the sample is proportional to the estimated concentration of biomarker in the wastewater at that time of day according to the diurnal biomarker pattern. Thus, the computer system analyzes a sample that is representative of actual human waste in the sewer system, thereby reporting concentrations of the biochemical indicator that exhibit less variability in the face of variation in the proportion of the wastewater flow originating from residential sources.

Wastewater samples collected by the wastewater sampling system can then be chemically tested to generate biomarker-calibrated biochemical indicator concentrations (e.g., in milligrams/milliliters). The computer system collects the biomarker-calibrated biochemical indicator concentrations; normalizes the biochemical indicator concentration based on the expected concentration of the biomarker in the sample; and adjusts the biochemical indicator concentration to account for the decay experienced by the biochemical indicator in the wastewater flow as the biochemical indicator traveled from an upstream source to the wastewater sampling system. In one implementation, the system determines a distribution of flow times for the water based on a simulation of wastewater flow in the upstream sewage network and adjusts the biochemical indicator concentration according to an estimate of the time wastewater from each upstream residence spent in the sewer system before reaching the wastewater sampling system. Furthermore, the computer system can account for the decay function (e.g., exponential, linear) of the biochemical indicator in wastewater.

The computer system can also calculate an estimate for the total biomarker-weighted flow through the sewage access point during the sampling period (e.g., 24 hours) in order to transform the adjusted biochemical indicator concentration to a biochemical indicator mass estimate (e.g., in milligrams/day/person) for the geographical region during the sampling period. Upon calculating the biochemical indicator mass estimate, the computer system can render a map including the geographical region served by the wastewater flow in association with the biochemical indicator mass.

Furthermore, the computer system can also transform the biochemical indicator mass estimate into an estimate of the equivalent number of dosages (if the biochemical indicator is a drug metabolite) of a drug or an equivalent number of cases (if the biochemical indicator is viral RNA) of a virus according to a predefined relationship between the biochemical indicator mass excreted by the human body for a given dosage of a drug or infection.

Once the computer system calculates a dosage or infection equivalent for the biochemical indicator or a biochemical indicator mass corresponding to a particular drug metabolite, the computer system can render a report indicating one of the above statistics in association with a map of the geographical region corresponding to the upstream sewer system from which the underlying samples were collected.

Additionally, the computer system can execute the above analysis for multiple biochemical indicators in wastewater in order to prompt and/or inform emergency response, health care, epidemic response, substance abuse, and law enforcement entities of drug usage and/or infection data that is accurate, hyper-local, and up-to-date when compared to longer term studies. The computer system can additionally provide prompts to these entities recommending particular responses based on the state of drug usage or infections in a particular geographical region. For example, the computer system can prompt an emergency response team to administer several additional doses of naloxone to overdose victims in the geographical region in response to detecting the presence of a drug metabolite corresponding to a more powerful opioid in the wastewater of the geographical region. In another example, the system can recommend a specific allocation of viral tests to a region based on a predicted number of infections within a region.

The computer system can analyze biochemical indicator data collected from multiple instances of the wastewater sampling system that are strategically deployed throughout a sewer network, such that each wastewater sampling system can sample wastewater from a primarily residential area with demographics representative of a larger area of a city. Thus, by integrating sewer network data, land use data, and demographic data to deploy the wastewater sampling system, the wastewater sampling system and computer system can indirectly provide a means to estimate illicit drug usage or otherwise monitor the public health of a population of a geographical region.

The computer system is primarily described herein with reference to the analysis of wastewater samples to estimate drug consumption/usage and/or viral infection. However, the computer system can analyze waterborne samples for any other application, such as broad public health monitoring.

### 3. Wastewater Sampling System

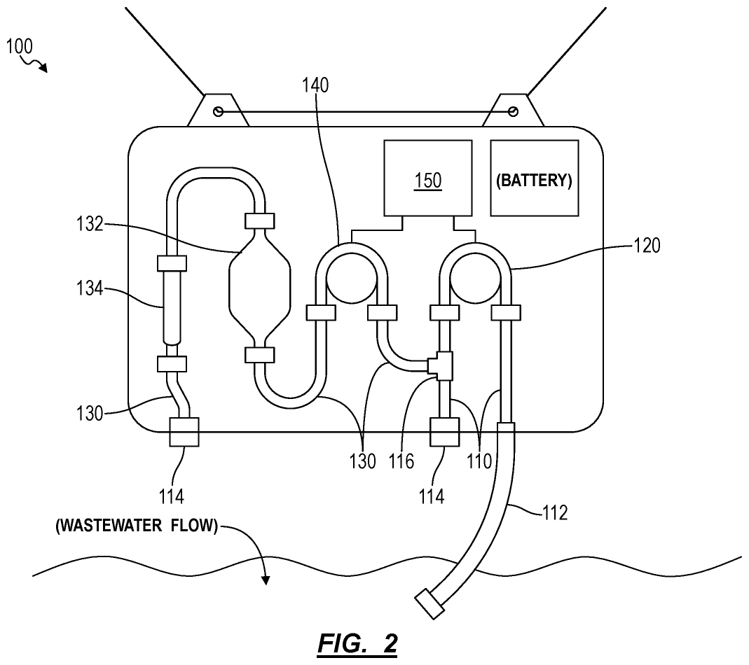

Generally, as shown in FIG. 2, the wastewater sampling system 100 (further described in U.S. patent application Ser. No. 16/528,465, which is incorporated by reference in its entirety) is an upstream wastewater sampling device configured to: be suspended above a wastewater flow proximal to a sewage access point (e.g. a manhole); periodically sample wastewater from the sewage flow in a precise dosage; filter solid waste from a wastewater sample; and draw the wastewater sample through a solid-phase extraction (hereinafter “SPE”) cartridge to extract dissolved biochemical indicators (e.g., drug metabolites or viral RNA) in the wastewater sample. The wastewater sampling system 100 also includes replaceable and serviceable components, which reduce the need for onsite servicing, thereby limiting deployment and redeployment costs while also ensuring consistent sampling. The wastewater sampling system 100 provides government entities or other interested parties the ability to sample wastewater upstream (in terms of the wastewater flow in the sewer network) from a wastewater treatment facility. By being deployed upstream, the wastewater sampling system 100 can be positioned closer to sources of residential wastewater, which may contain drug metabolites, drug components, other indicators of drug use, viral RNA, bacteria, or any other chemical or biological organism of interest from a particular residential area. These biochemical indicators may be consumed by bacteria in wastewater or otherwise decay in the wastewater flow before arriving at a water treatment facility. Therefore, the wastewater sampling system 100 is configured to collect a stable sample of wastewater containing detectable amounts of biochemical indicators and to separate these biochemical indicators from biological solid waste that may otherwise consume or contaminate these biochemical indicators, thereby enabling accurate, localized monitoring of drug consumption, infection rates, or any other detectable biochemical condition within a geographic region housing a human population. Furthermore, by streamlining deployment and redeployment to limit per unit cost and deployment time, many instances of the wastewater sampling system 100 can be distributed within a geographical region (e.g. an urban area or city) to collect high-resolution, localized, repeatable biochemical condition data for a population within this geographic region.

In addition to an SPE cartridge 134 for extracting biochemical indicators dissolved in the wastewater, the wastewater sampling system 100 also includes a solid waste filter 132, which traps a sample of the microbiome present in the wastewater. Therefore, in one implementation, the wastewater sampling system 100 can collect chemical samples in the SPE cartridge 134 for later analysis using mass spectrometry, as well as solid waste samples in the solid waste filter 132 for later analysis using DNA sequencing, such as 16S rDNA sequencing, to identify bacterial or viral species in the solid waste sample. Additionally or alternatively, the wastewater sampling system 100 can include a fluid repository 160 which can preserve a wastewater sample in a stable environment (i.e., without bacteria in the sample) for later analysis or as a “B sample” for verification of the original sample.

In one application of the wastewater sampling system 100, multiple instances of the wastewater sampling system 100 can be strategically deployed throughout a sewer network, such that each wastewater sampling device can sample wastewater from a primarily residential area with demographics representative of a larger area of a city. Thus, by analyzing sewer network data, land use data, and demographic data to deploy the wastewater sampling system 100, the wastewater sampling system 100 can enable estimation of illicit drug usage or otherwise monitor the public health of a population of a geographic region.

Once deployed at a sewer access point, the wastewater sampling system 100 can periodically initiate a sampling period, during which the wastewater sampling system 100 can execute sampling cycles to collect wastewater samples from the sewage flow. The wastewater sampling system 100 can utilize a sampling period of any duration such as a 24-hour period (i.e., daily sampling period), weekly sampling period, or monthly period. In one implementation, the wastewater sampling system 100 can also initiate the sampling period such that the end of the sampling period coincides with a scheduled pick-up or exchange of the SPE cartridge 134, solid waste filter 132, and/or the fluid repository 160, such that the sample may be analyzed shortly after it has been collected by the wastewater sampling system 100, thereby reducing degradation of the sample before analysis.

The wastewater sampling system 100 can also be configured for service or replacement after completion of a sampling period. Alternatively, the wastewater sampling system 100 can include multiple SPE filters, thereby enabling the wastewater sampling system 100 to execute collection of multiple wastewater samples over multiple sampling periods. In one application, the wastewater sampling system 100 defines a modular structure, which includes many of the replaceable components of the wastewater sampling system 100, such as the solid waste filter 132, the fluid repository 160, and the SPE cartridge 134, which can be replaced or exchanged. In another implementation, the system can include a sampling assembly 102, which includes the controller 150, pumps, the external casing, and other permanent and/or reusable components of the wastewater sampling system 100, and a replaceable filter kit 104, which can include all single use components of the system, such as the solid waste filter 132, the SPE cartridge 134, connective tubing, and/or the fluid repository 160. Therefore, a user servicing or collecting samples from the wastewater sampling system 100 can remove all replaceable components as a single unit.

The wastewater sampling system 100 includes a controller 150 arranged within the external casing of the wastewater sampling system 100 that initiates sample cycles and executes other functions. Alternatively, the system can interface with an external controller 150 to trigger particular functions of the wastewater sampling system 100.

One variation of the wastewater sampling system 100 includes an inlet tube 112, a first pump 120, a fluid repository 160, a second pump 140, a solid waste filter 132, and an SPE cartridge 134. In this variation, the system, via the controller 150, initiates intake of a wastewater sample from the sewage flow through the inlet tube 112 by activating the first pump 120. The first pump 120 draws the wastewater through the solid waste filter 132, which removes solid waste and bacteria from the wastewater. The wastewater sampling system 100 continues to draw wastewater from the inlet tube 112 via the controller 150 until a sampling volume has accumulated in the fluid repository 160. Once the sample volume of wastewater has accumulated, the wastewater sampling system 100 deactivates the first pump 120, thereby stopping the flow of wastewater through the solid waste filter 132 and into the fluid repository 160. The system then activates a second pump 140 to draw a sampling volume from the fluid repository 160 and through the SPE cartridge 134, thereby accumulating chemical samples within the SPE cartridge 134 for later analysis.

In another variation, the wastewater sampling system 100 can include two fluid circuits, a high flow circuit and a sampling circuit 130, along with the inlet tube 112, the first pump 120, the second pump 140, the solid waste filter 132, and the SPE cartridge 134. In this variation, the high-flow circuit 110 connects the inlet tube 112 to an outlet port 114, while the sampling circuit 130 is fluidly coupled to the high-flow circuit 110 via a junction 116 in the high-flow circuit 110. The sampling circuit 130 connects to the junction 116 in the high-flow circuit 110 and includes the solid waste filter 132 and the SPE cartridge 134. In this variation, during a sampling cycle, the wastewater sampling system 100 can activate the first pump 120, via controller 150 to draw wastewater water up from the sewage flow, into the wastewater sampling system 100 and back out of the outlet port 114. While the first pump 120 is activated and wastewater is flowing through the high-flow circuit 110, the wastewater sampling system 100 activates the second pump 140 to divert water from the high-flow circuit 110 into the sampling circuit 130 thereby collecting samples in both the solid waste filter 132 and SPE cartridge 134. In this variation, the second pump 140 is configured to divert precise volumes of wastewater from the high-flow circuit 110 in order to produce accurate chemical samples in the SPE cartridge 134.

In one implementation, the controller 150 is configured to execute additional functions such as: illuminating an LED indicator; timing the intake of the sampling volume; reading capacitive sensors arranged proximal to the fluid repository 160 to determine fluid levels; and/or reading values from a flowmeter. The wastewater sampling system 100 can also include a controller 150 configured to adjust the timing of the sampling period, the sampling interval, and the sampling cycle. Furthermore, the wastewater sampling system 100 can include a controller configured to adjust the flowrate of either the first pump 120 or the second pump 140 of the wastewater sampling system 100.

### 3.1 Deployment

The wastewater sampling system 100, can be deployed proximal to or above a wastewater flow in a sewer network. In one implementation the wastewater sampling system 100 can be deployed proximal to a sewer access point such as a manhole cover or manhole. The placement of the wastewater sampling system 100 proximal to a sewer access point allows for access to the system for servicing and also allows for some standardization in the deployment of the wastewater sampling system 100. In another implementation, the system can be deployed in a predominantly residential area (e.g. for monitoring illicit drug consumption), or a predominantly industrial area (e.g. for monitoring pollution levels in the wastewater). Ideally, the wastewater sampling system 100 is deployed at a location in the sewer network that typically experiences greater than a minimum level of sewage flow such that the wastewater sampling system 100 can extract a representative sample from the sewage flow. For example, if the minimum flow level at a specific location in the sewer network results in a water level too low for the wastewater sampling system 100 to retrieve a sample, the specific location may not be a suitable location for deployment of the wastewater sampling system 100. Likewise, the wastewater sampling system 100 is deployed at a location in a sewer network that experiences maximum flow levels that are not typically capable of displacing or otherwise damaging the wastewater sampling system 100.

The wastewater sampling system 100 can be configured for rapid deployment with tools available to public works employees or other government workers with access to a sewer network. In one implementation, the computer system can calculate a set of deployment locations (e.g., at sewer access points within a geographical region) for a set of wastewater sampling systems 100 in order to accomplish a target sampling coverage of the geographical region (i.e., a target proportion of the geographical region). Alternatively, the computer system can calculate a set of deployment locations of set of wastewater sampling system 100 such that the set of wastewater sampling systems 100 collected wastewater samples representing a target proportion of the population (e.g., based on census or other population data). Additionally, the computer system can calculate deployment locations such that the set of wastewater sampling systems 100 can collect wastewater samples demographically representing the population of a larger geographic region (e.g., a town or city), while only sampling a subregion or subpopulation of the larger geographical region.

Thus, the computer system analyses samples collected from the wastewater sampling system 100 (or a set of wastewater sampling systems 100)—deployed at a sewer access point upstream from a wastewater processing facility—according to the method S100. More specifically, the computer system can access a biochemical indicator concentration and a biomarker concentration from a wastewater sample collected by the wastewater sampling system 100 located upstream from a wastewater treatment facility.

### 3.2 Biochemical Conditions

Generally, the computer system accesses data collected from samples collected by the wastewater sampling system and processes these data according to the method S100 in order to detect the prevalence of a particular biochemical condition or a set of biochemical conditions (such as the prevalence of drug use, drug overdose, viral infection, bacterial infection) within a population served by the sewer system upstream from the wastewater sampling system 100. More specifically, the system can access, based on a wastewater sample collected by the wastewater sampling system 100 from a wastewater flow serving a population: a biochemical indicator concentration of a biochemical indicator characterized by a correlation of the biochemical indicator with a biochemical condition of interest. Thus, the computer system can estimate, based on this correlation, the prevalence of the biochemical condition in a population based on the presence of the biochemical indicator in wastewater.

For example, many illicit drugs such as opioids, amphetamines, etc., cause the human body to excrete (via urination) specific drug metabolites associated with these drugs. Therefore, the computer system can infer the usage rate of these drugs in a population based on the presence of the drug metabolites in the wastewater flowing through the sewer system serving the population. More specifically, in this application, the system can access, based on a wastewater sample collected by the wastewater sampling system 100 from a wastewater flow serving a population: a drug metabolite concentration of a drug metabolite characterized by a correlation of the drug metabolite with drug usage; and the biomarker concentration of the biomarker correlated with presence of human excreta.

In another example, a viral infection may cause a human body to excrete (via defecation) viral RNA and/or viral particles representative of these viruses. Therefore, the computer system can infer the infection rate (i.e. prevalence) of the particular virus throughout a population based on presence of RNA and/or viral particles—corresponding to this particular virus—in a wastewater sample drawn from a sewer system serving this population. More specifically, in this application, the computer system can access, based on the wastewater sample collected by the wastewater sampling system 100 from a wastewater flow serving a population: a viral particle concentration of a viral particle characterized by a correlation of the viral particle with viral infection; and the biomarker concentration of the biomarker correlated with presence of human excreta.

Thus, the computer system can detect any biochemical condition for which humans excrete some biological or chemical evidence of this condition within human excreta.

### 3.3 Biomarker Selection

Generally, the computer system can select a biomarker (e.g., urinary and/or fecal) with which to identify a diurnal sampling pattern of the wastewater sampling system 100 in order to normalize the concentration of the biochemical indicator. The computer system can select an appropriate biomarker with which to normalize the biochemical indicator concentration based on the particular biochemical condition of interest and the biochemical indicator of interest. More specifically, the computer system can select a biomarker originating from the same form of excreta as does the biochemical indicator, thereby providing an appropriate control for normalizing the concentration of the biochemical indicator.

For example, because drug metabolites excreted due to the consumption of opioids are shed primarily in urine, the computer system can select creatinine—which is consistently present in human urine—as a biomarker when estimating the prevalence of opioid consumption within a population from a wastewater sample. In another example, because viral RNA is primary shed in feces, the computer system can select a fecal biomarker when estimating the prevalence of viral infection within a population from a wastewater sample.

Thus, the computer system can access a urinary biomarker concentration correlated with presence of human urine and, based on the wastewater sample collected by the wastewater sampling system 100, normalize a drug metabolite concentration based on the urinary biomarker concentration to generate the normalized drug metabolite concentration. Alternatively, the computer system can access a fecal biomarker concentration correlated with the presence of human feces and, based on the wastewater sample collected by the wastewater sampling system 100, normalize a viral particle concentration based on the fecal biomarker concentration.

### 3.4 Sampling Setup

Upon or prior to deployment of the wastewater sampling system 100, the wastewater sampling system 100 can initially collect samples from discrete periods of a day. For example, the wastewater sampling system 100 can collect a sample for each hour of a day. The samples can then be analyzed for urinary biomarkers such as creatinine, urea, and/or uric acid and/or fecal biomarkers such as viral RNA from common gut viruses or common gut bacteria. These individual wastewater samples (i.e., calibration samples) can be compared to determine a diurnal pattern for the proportion of human waste (including urine and/or feces) that is typically present in the wastewater at the location of the wastewater sampling system 100. Thus, the computer system can estimate the diurnal biomarker pattern based on a set of calibration samples collected from the wastewater flow upstream from a wastewater treatment facility. The concentration of the biomarker in each sample, therefore, serves as a proxy for the proportion of human waste-related flow in the wastewater flow.

After observing a diurnal biomarker pattern, the wastewater sampling system 100 can then execute a sampling pattern wherein the wastewater sampling system 100 collects volumes of the wastewater proportional to the concentration of the biomarker detected within samples taken during each discrete time period within a day, as further described below.

In one implementation, the wastewater sampling system 100 executes a sampling pattern based on a day of the week in addition to the time of day in order to account for weekly variations in the human waste contribution to the wastewater flow.

In another implementation, the wastewater sampling system 100 or another device equipped with a flowmeter can measure and/or estimate the total daily wastewater flow at the location of the wastewater sampling system 100 (e.g., by taking an average over multiple days). Additionally or alternatively, the computer system can estimate the average daily wastewater by simulating fluid flow in the sewer network of the geographical region, as is further described below. The computer system can also collect estimates of the wastewater flow at the upstream location of the wastewater sampling system 100 by interfacing with a wastewater treatment facility.

In yet another implementation, the wastewater sampling system 100 can access a standard sampling pattern based on historical sample data collected from locations different from the current location of the wastewater sampling system 100.

### 3.5 Sampling Schedule

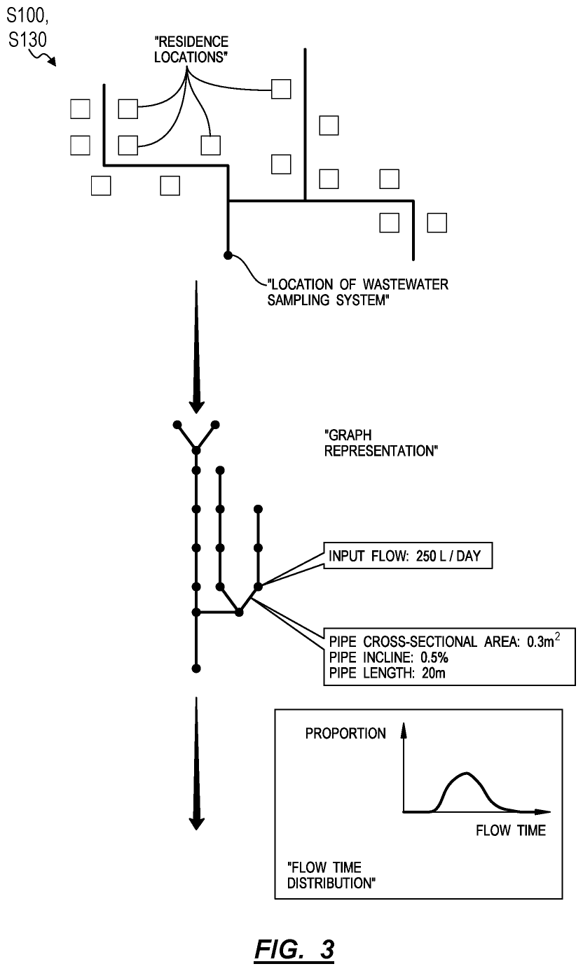

Generally, as shown in FIG. 3, upon detecting the diurnal biomarker patterns for the location of the wastewater sampling system 100, the wastewater sampling system 100 can be calibrated to adjust a normal periodic sampling schedule to a biomarker-calibrated schedule in order to sample the wastewater flow proportional to the concentration of the biomarker in the flow. Therefore, given a limited sample volume in the wastewater sampling system 100, the wastewater sampling system 100 can collect a sample that includes wastewater proportional to the amount of human waste in the wastewater flow at the time each part of the sample was retrieved from the wastewater flow. For example, given a total sample volume of 500 milliliters and a diurnal biomarker pattern that indicates 5% of the total amount of biomarker is present in the wastewater flow between 5 am and 6 am, the wastewater sampling system 100 collects 5% of its total sampling volume between 5 am and 6 am. Thus, when executing Blocks of the method S100, the computer system can access the biochemical indicator concentration and the biomarker concentration from a wastewater sample collected by the wastewater sampling system 100, the wastewater flow serving the population proportional to a diurnal biomarker pattern.

In one implementation, the wastewater sampling system 100 operates on a default sampling schedule where the wastewater sampling system 100 is actively sampling the wastewater flow for some proportion of the total sampling period (e.g., 20% of the sampling period). Additionally, the wastewater sampling system 100 can spread its sampling time evenly over the sampling period (e.g., a 24-hour period). Therefore, in order to modulate the volume of wastewater sampled by the wastewater sampling system 100 at any given time during a sampling period, the wastewater sampling system 100 can adjust the inlet flow rate during each sampling event. For example, if the wastewater sampling system 100 schedules a one-minute sampling event every five minutes (e.g., sampling 20% of the time), then the wastewater sampling system 100 can adjust the inlet flow rate during each one-minute sampling event in order to modulate the volume-proportion of the total sample for each discrete time interval of the diurnal pattern. Additionally or alternatively, the wastewater sampling system 100 can modify its sampling frequency and/or duration to modulate the amount of the sample collected during each discrete time interval of the diurnal pattern.

In one implementation, the wastewater sampling system 100 limits the time between sampling events during the sampling period to a threshold maximum sampling interval. The threshold maximum sampling interval can be selected such that the threshold maximum sampling interval approximates the time elapsed as wastewater content from a single toilet flush flows past the location of the wastewater sampling system 100. Thus, by executing the sampling schedule with a threshold maximum sampling interval, the wastewater sampling system 100 ensures that each toilet flush is included in at least on sample event.

### 3.6 Extracting Biochemical Indicator Concentration

The wastewater sampling system 100 includes a removable and/or replaceable SPE cartridge configured to extract dissolved biochemical indicators from water. Generally, the wastewater sampling system 100 pumps wastewater through the SPE cartridge, which extracts biochemical indicators and/or any other chemicals present in the wastewater sample for subsequent analysis via mass spectrometry, upon removal of the SPE cartridge from the wastewater sampling system 100.

In one implementation, the wastewater sampling system 100 includes an SPE cartridge with a stationary phase having analytes configured to bind with target functional groups of particular biochemical indicators or other target chemicals that may be present in the wastewater sample. However, the wastewater sampling system 100 can extract biochemical indicators or other target chemicals from the wastewater sample in any other way.

In another implementation, the wastewater sampling system 100 includes a set of multiple SPE cartridges and each SPE cartridge is included in its own sampling circuit in the wastewater sampling system 100. For example, each SPE cartridge in the set can be individually fluidly coupled to the fluid repository (e.g. via multiple tube junctions or diverter valves) and to the outlet port and each SPE cartridge can include an associated pump (further described below) to move wastewater samples through each SPE cartridge. Alternatively, the wastewater sampling system 100 can include a single pump coupled to a diverter valve to selectively pump water through each of the set of SPE cartridges. Therefore, the wastewater sampling system 100 can be capable of performing multiple twenty-four-hour sampling periods before being serviced or replaced.

Upon completing a sampling period, the SPE cartridge can be removed and sent to a lab for testing in order to extract biochemical indicator concentrations as a mass per volume of wastewater (e.g., in milligrams/milliliter) based on the total sampling volume and the mass of extracted biochemical indicator. The biochemical indicator concentrations are electronically recorded for access by the computer system.

In Block S110, the computer system can electronically access (e.g., via a network, or via local storage) the biomarker-calibrated biochemical indicator concentrations detected from the SPE cartridge and/or a concentration of the biomarker. More specifically, the computers system can access, based on the wastewater sample collected by the wastewater sampling system 100, a biochemical indicator concentration and a biomarker concentration. The computer system can collect biochemical indicator concentrations for a variety of drugs including naloxone, opioids, prescription drugs, or other illicit drug such as alcohols, amphetamines, barbiturates, benzodiazepines, and cannabinoids.

### 4. Biomarker Normalization

Generally, in Block S120, the computer system can: compare the biomarker concentration from the wastewater sample to an expected biomarker concentration collected from prior samples from the sample location of the wastewater sampling system 100; and normalize the biomarker concentration based on the biomarker concentration to generate a normalized biochemical indicator concentration. More specifically, the computer system can normalize the biochemical indicator concentration based on an inverse ratio of the biomarker concentration to the expected biomarker concentration to generate the normalized biochemical indicator concentration. Thus, the computer system can account for variations in the proportion of the wastewater flow including human waste. For example, rain may occur within the geographical region served by the sewer system in which the wastewater sampling system 100 is installed, thereby decreasing the proportion of the wastewater flow containing human waste (due to drainage of rainwater into the combined sewer system).

By normalizing the biochemical indicator concentration based on the biomarker concentration from the same sample, the computer system reduces variability in the biochemical indicator concentration by tying the biochemical indicator concentration to the actual amount of biomarker present in the wastewater sample.

### 5. Biochemical Indicator Decay Adjustment

Generally, in Block S130 and as shown in FIG. 3, the computer system can adjust the biochemical indicator concentration (or the biomarker-normalized biochemical indicator concentration) in order to account for decay of the biochemical indicator concentration in the wastewater flow (e.g., due to chemical interaction of the biochemical indicator with other species present in the wastewater or consumption of the biochemical indicator by bacteria in the wastewater) as the biochemical indicator travels from each residential wastewater source to the location of the wastewater sampling system 100 in order to produce an accurate estimate of the amount of the biochemical indicator introduced to the sewage network from reach residential wastewater source within the geographical region served by the wastewater flow at the sampling location, the computer system adjusts the biochemical indicator concentration based on decay characteristics of the biochemical indicator in sewage water and upstream wastewater flow to generate a decay-adjusted biochemical indicator concentration.

The computer system can simulate the duration of time wastewater has been in the sewer network before reaching the sampling location of the wastewater sampling system 100. Therefore, the computer system can access a map of the sewer network and/or utilities data to estimate the proportion of the wastewater flow from different locations within the geographical region. More specifically, the computer system can access a map of a sewer system upstream from a location of the wastewater sampling system 100, the map comprising a set of residence locations.

Given these initial conditions, the computer system can perform fluid mechanical simulation or a network flow simulation to estimate an amount of time wastewater has been in the sewer network upon reaching the sampling location of the wastewater sampling system 100. More specifically, the computer system can generate a simulation of the wastewater flow through the sewer system based on the map of the sewer system; and, for each residence location in the set of residence location, estimate a flow time in the distribution of flow times from the residence location to the location of the wastewater sampling system 100 based on the simulation of the wastewater flow.

In one implementation, the computer system defines each residential location represented in the sewer map as a node in a graph representation and defines each edge between these nodes based on the wastewater flow within the sewer system connecting these residential locations. Each edge represents the characteristics of the sewer system between the two residential locations and can represent variables such as the cross-sectional area of the pipe and the inclination of the pipe. Thus, the system can estimate the velocity of wastewater within the section of the sewer system represented by the edge based on the volumetric flow rate through the section of the sewer system, the inclination of the section of the sewer system, and the cross-sectional area of the section of the sewer system.

To estimate the volumetric flow rate through each section of the sewer system upstream from the location of the wastewater sampling system 100, the computer system can average levels of daily water usage from each residential location, or the system can assume an average water consumption (i.e., wastewater input into the sewer system) for each residential location in the upstream sewer system based on historical data. For each node represented in the graph, the computer system can sum the volumetric flow rate of wastewater from all prior nodes in the graph, thereby representing the accumulation of wastewater in the sewer system downstream for each branch of the sewer system.

Once the computer system has estimated the volumetric flow rate at each point in the sewer system upstream from the location of the wastewater sampling system 100, the computer system can determine the speed of the wastewater flow for each edge in the graph based on the size and/or incline of the pipes in each corresponding section of the sewer system. Thus, the system can simulate a flow time for wastewater entering the upstream sewer system from any residential location in the sewer system.

In one implementation, the computer system can estimate an average flow time for the wastewater sample based on upstream characteristics of the wastewater flow as represented in the simulation of the sewer system. More specifically, the computer system can adjust the biochemical indicator concentration based on the decay characteristics of the biochemical indicator in wastewater and the average flow time for the wastewater sample to generate the decay-adjusted biochemical indicator concentration.

Alternatively, in order to better account for nonlinear decay functions of many biochemical indicators, the computer system can estimate a distribution of flow times for components of the wastewater flow originating from each residential location represented in the map of the sewer system. For example, the computer system can sum the flow time from each node in the above-described graph representation of the sewer system to the location of the wastewater sampling system 100. Thus, the computer system can estimate a flow time distribution for the wastewater sample based on upstream characteristics of the wastewater flow.

Upon estimating the flow time for the wastewater flow, the computer system can calculate an adjustment factor for a biochemical indicator concentration that accounts for the decay characteristics of the particular biochemical indicator. In one implementation, the computer system collects decay functions for each biochemical indicator analyzed by the computer system. The decay functions relate the time spent in the sewer network to the proportion of biochemical indicator remaining in the wastewater. The computer system can access theoretically derived decay functions or empirically estimated decay functions. In one implementation, the computer system can incorporate empirical data from the sewer network of interest (e.g., samples taken at various points along a pipe in the sewer network) in order to estimate a more accurate decay function for each biochemical indicator.

The computer system can evaluate the decay function for a biochemical indicator according to the average flow time of the wastewater flow. Alternatively, the system can calculate an integral average of the decay function of the biochemical indicator over the distribution of flow times of the wastewater flow. Thus, the computer system can adjust the biochemical indicator concentration based on the decay characteristics of the biochemical indicator in wastewater and the flow time distribution for the wastewater sample to generate the decay-adjusted biochemical indicator concentration. Thus, the computer system can: access a decay function of the biochemical indicator in wastewater; and adjust the biochemical indicator concentration based on the decay function of the biochemical indicator in wastewater to generate the decay-adjusted biochemical indicator concentration.

In either case, the computer system generates a decay factor (e.g., proportional decay) experienced by the biochemical indicator sample between the average source in the sewer network and the sampling location. The computer system can then divide the biochemical indicator concentration by the decay factor to calculate a decay-adjusted biochemical indicator concentration.

### 6. Mass Estimation

Generally, in Block S140 the computer system multiplies the decay-adjusted biochemical indicator concentration by an effective flow volume (i.e., an estimated total flow volume for the sampling period) of the wastewater flow at the location of the wastewater sampling system to generate a biochemical indicator mass estimate for the population within the geographical region. Thus, the decay-adjusted biochemical indicator concentration (e.g., expressed in milligrams/milliliter/day) can be converted to a total biochemical indicator mass for the geographical region (e.g., expressed in milligrams/day). The computer system can calculate an effective daily flow volume via a flow simulation or as an average flow volume based on the flow rate of the wastewater flow over the sampling period, or as an actual measure of the total flow volume during the sampling period.

In one implementation, the computer system calculates the effective flow volume based on a number of residences served by the sewer system upstream from the location of the wastewater sampling system 100 and the average water usage for these residences. For example, if there are 200 residences served by the sewer system upstream from the location of the wastewater sampling system 100 and these residences use an average of 60 liters of water a day, then the computers system can calculate the effective flow volume to be 12,000 liters. Additionally, the computer system can estimate the proportion of the wastewater sample that is derived from residential sources based on the biomarker concentration derived from the wastewater sample and adjust the effective daily flow rate based on this proportion. For example, the computer system can access an expected proportion of the biomarker in a wastewater sampling including only residential wastewater and adjust the effective flow volume proportional to the ratio of the actual biomarker concentration in the wastewater sample to the expected biomarker concentration in the wastewater sample.

### 7. Per-Person Mass Estimation

Generally, the computer system can convert the total biochemical indicator mass for the population within the geographical region to biochemical indicator mass per person (e.g., expressed in milligrams/day/1000 people) estimate by dividing the total biochemical indicator mass by an estimated population of the geographical region. In one example, the computer system can access census data for the geographical region in order to estimate the population of the region. Alternatively, the computer system can estimate the total population contributing human waste to the wastewater flow during the sampling period for the current sample by calculating (according to Blocks of the method S100 described above) the total mass of the biomarker and correlating the total mass of the biomarker to an estimated population for the geographical region based on the average mass of the biomarker produced per-person-per-day.

In yet another implementation, the computer system can associate a census population estimate for the geographical region with an average biomarker mass and adjust a current population based on the census population estimate and a deviation of the biomarker mass in the sample from the average biomarker mass.

Additionally or alternatively, the computer system can estimate a population size for the geographic region based on the genetic diversity of the microbiome collected by the solid waste filter of the wastewater sampling system 100. More specifically, the computer system can calculate a measure of genetic diversity for a microbiome that was collected concurrently with the biochemical indicator mass and, based on a correlation between the genetic diversity metric and population size, estimate the population that contributed to the biochemical indicator mass.

### 8. Prevalence Estimation

Generally, in Block S150, the computer system converts the biochemical indicator mass estimate to a prevalence of the biochemical condition corresponding to the biochemical indicator based on dosage and/or infection rate information for the biochemical condition. More specifically, the computer system can estimate a prevalence of the biochemical condition in the population based on the correlation of the biochemical indicator to the biochemical condition and the biochemical indicator mass estimate. Thus, the computer system leverages known prevalence data on human metabolism, viral shedding, or bacteria shedding based on the biochemical condition of interest to determine a mass of the biochemical indicator corresponding to a single the prevalence of the biochemical condition. The computer system can then transform the mass per-person-per-day of the biochemical indicator to per-person per-day the prevalence, which may be significantly more meaningful in communicating with various healthcare-related entities.

In one implementation, for applications in which the biochemical condition is a drug and the dosage information for the drug is not available, the computer system can access stoichiometric relationships in the metabolic pathway for the drug to estimate the dosage equivalent for the drug.

Additionally or alternatively, the computer system can transform the dosage equivalent per-person of the drug to a morphine-dosage equivalent based on a relationship between the drug and morphine. For example, the computer system can access information indicating that a single dose of carfentanil is equivalent to 10,000 doses of morphine. Thus, if the computer system calculates that a single dose of carfentanil was taken within the geographical region during the sampling period, the computer system can convert the single dose to 10,000 morphine-equivalent doses. The computer system can access a predetermined table including morphine dose conversion factors for set of common opioid drugs.

### 9. Demographic Extrapolation

In one implementation, the computer system can analyze biomarker-calibrated samples collected at a number of wastewater sampling systems 100 distributed in different sampling locations (at sewer access points) across the sewer network of a larger region in order to estimate the prevalence of the biochemical condition for a larger population based on a demographic extrapolation between the geographic regions being sampled by each wastewater sampling system 100 and the larger region. Therefore, the computer system can extrapolate a biochemical indicator mass (or any other metric described above) corresponding to each geographical region within a larger region to estimate a total biochemical mass for the larger region.

Furthermore, the computer system can execute Blocks of the method S100 to estimate a biochemical indicator mass for each of a set of biochemical conditions, wherein each biochemical indicator mass corresponds to a known biochemical condition and is collected in a biomarker-calibrated sample collected by the wastewater sampling system 100. Additionally, the computer system can perform metanalyses of biochemical the prevalence patterns (further described below), for example, by comparing the prevalence rates between drugs in the set of detectable drugs or by comparing the prevalence rates between viral infections.

### 10. Report Generation

Generally, the computer system can render the biochemical indicator mass estimate in association with the geographic area; and the computer system can render the prevalence of the corresponding biochemical condition in association with the geographic area. More specifically, the computer system can render the prevalence of the biochemical condition in the population in association with a geographical region corresponding to the population.

Thus, the computer system can render a map of a larger geographical region and highlight specific sub-regions according to the presence of a wastewater sampling system 100 collecting samples from a wastewater flow serving the region. Thus, the computer system can highlight each geographical region for which the computer system calculates prevalence statistics in a map of a larger geographical region. Furthermore, the computer system can annotate each highlighted geographical region according to the prevalence statistics most recently calculated for the geographical region. The computer system can indicate one or more prevalence statistics (e.g., for a set of detected biochemical conditions) using color legends, numerical annotation, or any other graphical technique. In one implementation, the computer system can also render a map overlaid with a map of the sewer network for the geographical region. Additionally, the computer system can render the location of wastewater sampling system 100 in the sewer network on the map of the larger geographical region.

In one implementation, the computer system can render any of the aforementioned the prevalence metrics described such as the total biochemical indicator mass for a region, the biochemical indicator mass per-person in the region, and/or the prevalence per-person within the region.

Furthermore, in one implementation, the computer system can render comparisons between the usage rates of various drugs. In one example, the computer system can render a ratio of naloxone doses to the number morphine-equivalent doses to identify an overdose ratio in the geographical region.

In another implementation, the computer system renders other secondary statistics such as the rate of change of another drug usage statistic when compared with a preceding drug usage report. Additionally or alternatively, the computer system can compare the drug usage of a particular geographical region with all geographical regions being analyzed by the computer system.

In yet another implementation, the computer system can compare drug usage statistics estimated by the computer system to data provided by external sources. In one example, the computer system renders the number of naloxone doses estimated within the geographical region and the number of overdoses recorded by health services within the geographical region to indicate a number of unreported overdoses in the region.

In yet another implementation, the computer system can detect correlations between the usage of two different drugs within the geographical region and generate a report indicating the correlation may be indicative of a popular drug mixture in the geographical region.

In another implementation, the computer system can generate a report ranking drugs detected within the geographical region based on each drug's correlation with overdoses in the geographical region (e.g., as measured by a detected number of naloxone doses or a reported overdose statistics).

In applications of the computer system in which the computer system estimates the prevalence of a viral infection, the computer system can generate a report indicating the number of cases of a virus reported for the geographical region (e.g., by the government or public health authorities of the geographical region) compared to the estimated number of cases of the virus based on the prevalence estimate calculated by the computer system. Thus, the computer system can display an estimate of a number of undetected cases within a geographical region.

However, the computer system can render any other drug usage or related statistic in the report including the map of the geographical region.

### 11. Prompting Emergency Responses

Generally, the computer system can prompt an emergency response adjustment for the geographical region based on the prevalence of the biochemical condition. For example, the computer system can automatically communicate with healthcare-related entities such as hospitals and health policy entities to recommend new resource allocations in response to increases in drug usage and/or rates of infection as detected by the computer system according to Blocks of the method S100 described above. Drug-specific and infectious-disease-specific responses are further described below.

### 11.1 Prompting Drug-Specific Responses

In one variation, the computer system can guide various healthcare- and law-enforcement-related entities in executing various drug-related responses. More specifically, the computer system can repeatedly (e.g., daily, weekly, monthly) perform Blocks of the method S100 to estimate drug usage in a geographical region and prompt a drug-related entity to perform a drug-specific response for the geographical region based on up-to-date drug usage information for the geographical region. Therefore, emergency response entities, substance abuse prevention entities, and law enforcement entities can more effectively provide services to the population of the geographical region.

In one implementation, the computer system is configured to communicate directly with an emergency healthcare provider (e.g., a paramedic service) that responds to drug overdoses within the geographical region. The computer system can therefore notify the healthcare provider of up-to-date drug patterns in the geographical region. Additionally or alternatively, the system can generate a decision tree or response hierarchy for paramedics in the geographical region based on a ranking of the most prevalent drugs recently used within the geographical region. For example, the computer system can recommend a number of naloxone doses to administer to an unresponsive patient within the geographical region and, in the case of significant non-opioid drug use, recommend other treatment options prior to or subsequent to the administration of naloxone to the patient.

In another implementation, the computer system can transmit an alert to law enforcement entities, upon detecting greater than a threshold level of chemical byproducts of drug production within a region, thereby indicating the presence of illicit drug manufacturing within the region.

In yet another implementation, the computer system can communicate with substance abuse prevention entities to recommend locations within the geographical region for drug prevention outreach and education and/or rehabilitation clinics, thereby improving access for communities struggling with drug addiction and over-use.

However, the computer system can provide reports or prompt specific drug-related actions with any interested entity and in any other way.

### 11.2 Prompting Infectious-Disease-Specific Responses

In another variation, the computer system can generate guidance for responding to a transmissible disease epidemic effecting a population and serve this guidance to a healthcare or public-health-related entity. More specifically, the computer system can repeatedly (e.g., daily, weekly, monthly) execute Blocks of the method S100 to: estimate the number of infections in a geographical region; identify healthcare entities serving the geographical area based on map data; calculate a response adjustment for the geographical region based on the prevalence of the biochemical condition in the population; and prompt the response adjustment for the geographical region, such as executing a disease-specific response for the geographical region based on up-to-date infection rates for the geographical region. Thus, healthcare-related entities can reallocate treatment resources such as therapeutic devices, drugs, and/or personal protection equipment based on disease prevalence calculated by the computer system.

In order to prompt response adjustments for healthcare-related entities the computer system can identify the geographical region served by the wastewater flow from which the wastewater sampling system 100 is sampling based on a map of a sewer system. Once the computer system identifies this geographical region, the computer system can access map data to identify healthcare-related entities (e.g., such as hospitals, epidemiologists, contact tracing systems) operating near (e.g., within a threshold distance of) the geographical region.

Once the computer system identifies a set of healthcare-related entities, the computer system can calculate a response adjustment for the set of healthcare-related entities based on the type of healthcare-related entity, the particular biochemical condition for which the computer system has estimated a prevalence, and/or the characteristics of the population within the geographical region (e.g., known preexisting health conditions present within the population.)

In one implementation, the computer system can calculate a predicted number of hospitalizations within the geographical region based on the disease prevalence calculated by the computer system, the hospitalization rate of the disease, and known preexisting health conditions affecting the population within the geographical area.

In another implementation, the computer system can prompt healthcare providers within the geographical region to increase their supply of PPE in response to an increase in the predicted hospitalization within the geographical region. Additionally or alternatively, the computer system can prompt healthcare providers within the geographical region to increase their supply of therapeutic drugs or therapeutic devices (e.g., ventilators) in response to an increase in the predicted number of hospitalizations within the geographical region.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A method for estimating the prevalence of a biochemical condition in a population:
accessing a biochemical indicator concentration of a biochemical indicator correlated with the biochemical condition, the biochemical indicator concentration derived from a wastewater sample collected from a wastewater flow serving the population by a wastewater sampling system;
accessing a biomarker concentration of a biomarker correlated with presence of human excreta, the biomarker concentration derived from the wastewater sample;
normalizing the biochemical indicator concentration based on the biomarker concentration to calculate a normalized biochemical indicator concentration;
adjusting the normalized biochemical indicator concentration based on decay characteristics of the biochemical indicator in wastewater to generate a decay-adjusted biochemical indicator concentration;
calculating a biochemical indicator mass estimate for the population based on the decay-adjusted biochemical indicator concentration and an effective flow volume of the wastewater flow; and
estimating the prevalence of the biochemical condition in the population based on the correlation of the biochemical indicator to the biochemical condition and the biochemical indicator mass estimate.

2. The method of claim 1, wherein accessing the biochemical indicator concentration comprises accessing the biochemical indicator concentration from the wastewater sample collected from the wastewater flow serving the population by the wastewater sampling system at a sampling location upstream from a wastewater treatment facility.

3. The method of claim 2, further comprising:
identifying a geographical region served by a sewer system upstream from the sampling location based on a map of the sewer system; and
rendering the prevalence of the biochemical condition in the population in association with a depiction of the geographical region.

4. The method of claim 3, further comprising:
identifying emergency response entities serving the geographical area based on map data; and
calculating an emergency response adjustment for the geographical region based on the prevalence of the biochemical condition in the population; and
prompting the emergency response adjustment for the geographical region.

5. The method of claim 1:
wherein accessing the biochemical indicator concentration comprises accessing the biochemical indicator concentration of the biochemical indicator, the biochemical indicator originating from a first form of human excreta; and
wherein accessing the biomarker concentration comprises accessing the biomarker concentration of the biomarker, the biomarker originating from the first form of human excreta.

6. The method of claim 1:
wherein accessing the biochemical indicator comprises accessing a drug metabolite concentration of a drug metabolite characterized by a correlation of the drug metabolite with drug usage, the drug metabolite concentration derived from the wastewater sample collected from the wastewater flow serving the population by the wastewater sampling system;
wherein normalizing the biochemical indicator concentration based on the biomarker concentration to generate the normalized biochemical indicator concentration comprises normalizing the drug metabolite concentration based on the biomarker concentration to generate the normalized drug metabolite concentration;
wherein adjusting the normalized biochemical indicator concentration based on decay characteristics of the biochemical indicator in wastewater to generate the decay-adjusted biochemical indicator concentration comprises adjusting the normalized drug metabolite concentration based on decay characteristics of the drug metabolite in wastewater to generate a decay-adjusted drug metabolite concentration;
wherein calculating the biochemical indicator mass estimate for the population based on the decay-adjusted biochemical indicator concentration and the effective flow volume of the wastewater flow comprises calculating a drug metabolite mass estimate for the population based on the decay-adjusted drug metabolite concentration and the effective flow volume of the wastewater flow; and
wherein estimating the prevalence of the biochemical condition in the population based on the correlation of the biochemical indicator to the biochemical condition and the biochemical indicator mass estimate comprises estimating the prevalence of the drug usage in the population based on the correlation of the drug metabolite to a dose of a drug and the drug metabolite mass estimate.

7. The method of claim 6:
wherein accessing the biomarker concentration comprises accessing a urinary biomarker concentration of a urinary biomarker correlated with presence of human urine, the urinary biomarker concentration derived from the wastewater sample; and
wherein normalizing the drug metabolite concentration based on the biomarker concentration to generate the normalized drug metabolite concentration comprises normalizing the drug metabolite concentration based on the urinary biomarker concentration to generate the normalized drug metabolite concentration.

8. The method of claim 1:
wherein accessing the biochemical indicator concentration comprises accessing a viral particle concentration of a viral particle correlated with a viral infection, the viral particle concentration derived from the wastewater sample collected from the wastewater flow serving the population by the wastewater sampling system;
wherein normalizing the biochemical indicator concentration based on the biomarker concentration to generate the normalized biochemical indicator concentration comprises normalizing the viral particle concentration based on the biomarker concentration to generate the normalized viral particle concentration;
wherein adjusting the normalized biochemical indicator concentration based on decay characteristics of the biochemical indicator in wastewater to generate the decay-adjusted biochemical indicator concentration comprises adjusting the normalized viral particle concentration based on decay characteristics of the viral particle in wastewater to generate a decay-adjusted viral particle concentration;
wherein calculating the biochemical indicator mass estimate for the population based on the decay-adjusted biochemical indicator concentration and the effective flow volume of the wastewater flow comprises calculating a viral particle mass estimate for the population based on the decay-adjusted viral particle concentration and the effective flow volume of the wastewater flow; and
wherein estimating the prevalence of the biochemical condition in the population based on the correlation of the biochemical indicator to the biochemical condition and the biochemical indicator mass estimate comprises estimating the prevalence of the viral infection in the population based on the correlation of the viral particle to viral shedding of an infected human and the viral particle mass estimate.

9. The method of claim 8:
wherein accessing the biomarker concentration comprises accessing a fecal biomarker concentration of a fecal biomarker correlated with presence of human feces, the fecal biomarker concentration derived from the wastewater sample;
wherein normalizing the viral particle concentration based on the biomarker concentration to generate the normalized viral particle concentration comprises normalizing the viral particle concentration based on the fecal biomarker concentration to generate the normalized viral particle concentration.

10. The method of claim 1, further comprising:
estimating a number of residences upstream from the wastewater sampling system;
accessing an average water consumption of each residence in the number of residences;
calculating the effective flow volume based on the number of residences and the average water consumption of each residence in the number of residences; and
adjusting the effective flow volume based on the biomarker concentration and an expected biomarker concentration.

11. A method for estimating the prevalence of a biochemical condition in a population:
accessing a biochemical indicator concentration of a biochemical indicator correlated with the biochemical condition, the biochemical indicator concentration derived from a wastewater sample collected from a wastewater flow serving the population by a wastewater sampling system;
accessing a biomarker concentration of a biomarker correlated with presence of human excreta, the biomarker concentration derived from the wastewater sample;
normalizing the biochemical indicator concentration based on the biomarker concentration to generate a normalized biochemical indicator concentration;
calculating a biochemical indicator mass estimate for the population based on the normalized biochemical indicator concentration and an effective flow volume of the wastewater flow; and
estimating the prevalence of the biochemical condition in the population based on the correlation of the biochemical indicator to the biochemical condition and the biochemical indicator mass estimate.

12. The method of claim 11, wherein accessing the biochemical indicator concentration comprises accessing the biochemical indicator concentration of the biochemical indicator correlated with the biochemical condition, the biochemical indicator concentration derived from the wastewater sample collected proportional to a diurnal biomarker pattern

13. The method of claim 12, further comprising estimating the diurnal biomarker pattern based on a set of calibration samples collected from the wastewater flow at a location of the wastewater sampling system upstream from a wastewater treatment facility.

14. The method of claim 11, wherein normalizing the biochemical indicator concentration based on the biomarker concentration to generate the normalized biochemical indicator concentration comprises normalizing the biochemical indicator concentration based on an inverse ratio of the biomarker concentration to the expected biomarker concentration to generate the normalized biochemical indicator concentration.

15. The method of claim 11:
further comprising adjusting the normalized biochemical indicator concentration based on decay characteristics of the biochemical indicator in wastewater to generate a decay-adjusted biochemical indicator concentration; and
wherein calculating the biochemical indicator mass estimate for the population based on the normalized biochemical indicator concentration and the effective flow volume of the wastewater flow comprises calculating the biochemical indicator mass estimate for the population based on the decay-adjusted biochemical indicator concentration and the effective flow volume of the wastewater flow.

16. A method for estimating a prevalence of a biochemical condition in a population:
accessing a biochemical indicator concentration of a biochemical indicator correlated with the biochemical condition, the biochemical indicator concentration derived from a wastewater sample collected from a wastewater flow serving the population by a wastewater sampling system;
adjusting the biochemical indicator concentration based on decay characteristics of the biochemical indicator in wastewater to generate a decay-adjusted biochemical indicator concentration;
calculating a biochemical indicator mass estimate for the population based on the decay-adjusted biochemical indicator concentration and an effective flow volume of the wastewater flow; and
estimating the prevalence of the biochemical condition in the population based on the correlation of the biochemical indicator to the biochemical condition and the biochemical indicator mass estimate.

17. The method of claim 16:
further comprising estimating an average flow time for the wastewater sample based on upstream characteristics of the wastewater flow; and
wherein adjusting the biochemical indicator concentration based on the decay characteristics of the biochemical indicator in wastewater to generate the decay-adjusted biochemical indicator concentration comprises adjusting the biochemical indicator concentration based on the decay characteristics of the biochemical indicator in wastewater and the average flow time for the wastewater sample to generate the decay-adjusted biochemical indicator concentration.

18. The method of claim 16:
further comprising estimating a flow time distribution for the wastewater sample based on upstream characteristics of the wastewater flow; and
wherein adjusting the biochemical indicator concentration based on the decay characteristics of the biochemical indicator in wastewater to generate the decay-adjusted biochemical indicator concentration comprises adjusting the biochemical indicator concentration based on the decay characteristics of the biochemical indicator in wastewater and the flow time distribution for the wastewater sample to generate the decay-adjusted biochemical indicator concentration.

19. The method of claim 18, wherein estimating a flow time distribution for the wastewater sample based on upstream characteristics of the wastewater flow comprises:
accessing a map of a sewer system upstream from a location of the wastewater sampling system, the map comprising a set of residence locations;
generating a simulation of the wastewater flow through the sewer system based on the map of the sewer system; and
for each residence location in the set of residence locations, estimating a flow time in the distribution of flow times from the residence location to the location of the wastewater sampling system based on the simulation of the wastewater flow.

20. The method of claim 16, wherein adjusting the biochemical indicator concentration based on decay characteristics of the biochemical indicator in wastewater to generate the decay-adjusted biochemical indicator concentration comprises:
accessing a decay function of the biochemical indicator in wastewater; and
adjusting the biochemical indicator concentration based on the decay function of the biochemical indicator in wastewater to generate the decay-adjusted biochemical indicator concentration.
